(19) United States
(12) Patent Application Publication
(10) Pub. No.: US 2014/0142501 A1
(43) Pub. Date: May 22, 2014

(54) Bra With Integrated Breast Pump

(71) Applicants: Leanne Christa Clark, Saint Louis, MO (US); Nicholas Christopher Baldi, Saint Louis, MO (US)

(72) Inventors: Leanne Christa Clark, Saint Louis, MO (US); Nicholas Christopher Baldi, Saint Louis, MO (US)

(21) Appl. No.: 13/680,480

(22) Filed: Nov. 19, 2012

(51) Int. Cl. A61M 1/06

(52) U.S. Cl. CPC A61M 1/06
USPC 604/74

(57) ABSTRACT

A breast pump brassiere, designed to provide hands free experience for the mother when collecting mother's milk, and which integrates various operative components in the category of a micro pump, at least one suction cup, and a milk reservoir, preferably within each cup, to provide for hands free collection of expressed milk, as desired and required by the mother for use in the feeding of her infant.

### FIELD OF THE INVENTION

This invention relates to the development of a brassiere, and more specifically one that incorporates the mechanics for functioning as a breast pump for the new mother.

### BACKGROUND OF THE INVENTION

Obviously, brassieres have been available for many, many years. These types of garments have been designed to lift, separate, and restrain the breast of women since as early as 2000 b.c. Today, the bra is not only designed for support of the breast, it is also applied for fashion, and some bras have facilitated access to allow the new mother to breast feed its infant.

### SUMMARY OF THE INVENTION

The concept of this invention contemplates the modifications to a brassiere, and more specifically integrates into its structure various suction cups, a separate reservoir, that may locate either integrally within the structure of the bra, and obviously removable therefrom, or it may locate externally. Furthermore, the invention contemplates the use of a micro pump that is designed to develop a lower vacuum at the location of the suction cup, in order to induce the flow of milk from the mother's breast, and its collection within a reservoir, for subsequent usage.

The concept of this invention provides a hands free breast pump bra that is design to provide minimum participation of the working mother, when it is necessary to drain milk from the breast that can be subsequently used. The concept of this invention incorporates the pump within a bra, one that is designed and formed of cotton, elastic cloth, or even nylon, and may have an underwire for support. The bra contains a small micro pump, located between the breasts, within the structure of the brassiere, and embodied within a small pouch that it can easily be removable. The front of the bra has a flap that is openable, and within each flap is tubing that is located and which may be removable and washable. In the center of the flap directly over the areola is where a suction cup locates, for one or the other of the bra coverings. Obviously, this is a special time in a women's life, and it is important to have a comfortable nursing bra for application. As a result, there exists a need, which is supplied herein, for a hands free breast pump bra that would make life easier for the nursing mother, while expressing milk for her newborn. Furthermore, there exist the need for such a bra that is washable, practical, where its various components can be easily removed for cleaning and processing, but which bra can be worn either within or outside the home, for the convenience of the nursing mother, and readily available for usage, for the collection of fluids, when required and needed.

This bra acts as a breast pump that consists of a micropump located in a small pouch between the breast, or may be clipped onto pants pocket, it is removable when not in use, and the pump may be AC/DC operative, which means that a battery can be used for charging and energizing the pump, and preferable the charge will last at least approximately 6 hours, so as to be readily available for usage when demand requires. All tubing within the bra/pump is also removable. There are designed small semicircular shaped pouches that catch the mother's milk on either side of the breast cup, for collection. When through pumping, the mother will remove the pump so she can then go about her daily chores, in comfort, and the pump will be easily accessible so she can reuse it, when time requires.

It is, therefore, the principal object of this invention to provide a brassiere that integrates all the various structures that may be required for achieving the pumping, collection, and providing the capability of storage, of breast milk, for the new mother, while nursing her infant.

Another object of this invention is to provide a bra that may integrate all of the structures as previously defined, but yet function normally as a brassiere, as when not in usage.

Still another object of this invention is to provide a brassiere that integrates all the various structural components that may be needed for pumping and collecting mother's milk, but which all can be easily removed, from the brassiere, when storage of the milk is required, and cleaning of the various components is necessary.

Another object of this invention is to provide a brassiere integrating all the structures necessary for the collection of breast milk, at any time, regardless of where the mother may be located, whether at home, at work, driving, or even while shopping or performing other chores.

These and other objects may become more apparent to those skilled in the art upon review of the summary of the invention as provided herein, and upon undertaken a study of the description of its preferred embodiment, in view of the drawings.

### BRIEF DESCRIPTION OF THE DRAWINGS

In referring to the drawings,

FIG. 1 shows a bra, structured for use for the purposes of this current invention and which has built in technology to function as a breast pump;

FIG. 2 shows the brassiere of FIG. 1, but disclosing the location of the various suction cups, micropump, and the various fluid reservoirs; also shows a suction cup that is integrated, one into each of the cups of the shown brassiere; shows a micropump, that generally locates centrally of the shown brassiere, and which is designed for connection with tubing, from the selective cups, for use for inducing suction of breast milk through the associated suction cup, and for its transfer to a fluid reservoir; show the configuration in length of the various tubing's that may be applied between the suction cup, the micropump, and the fluid reservoir.

FIG. 3 shows a reservoir, one of which may be located within each of the cups of the shown brassiere, and which may incorporate tubing that interconnects each reservoir with its associated suction cup; and shows one of the suction cups for this development.

FIG. 4 discloses how the suction cups locate within in a pocket formed in each cup of the bra.

### DESCRIPTION OF THE PREFERRED EMBODIMENT

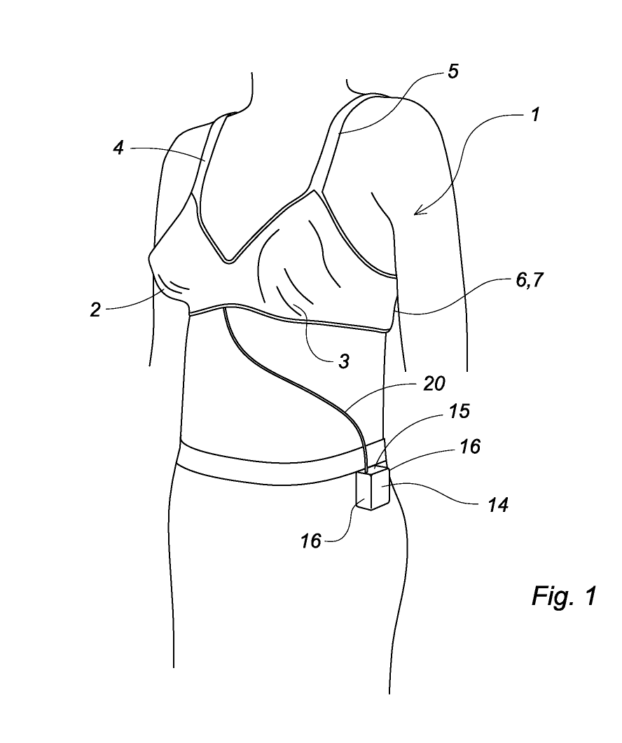

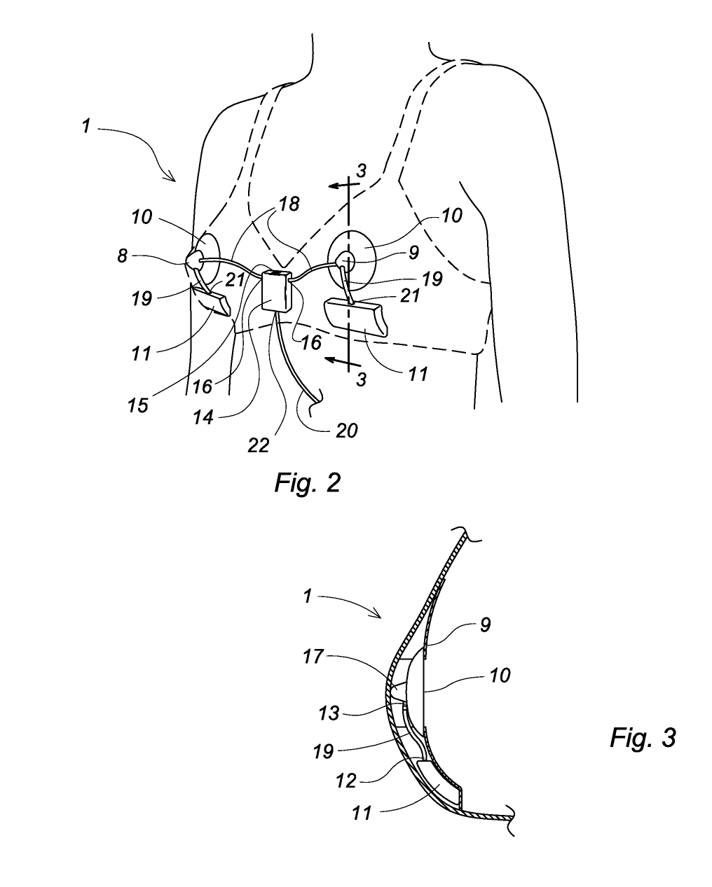

In referring to the drawings, and in particular FIGS. 1, therein is shown the bra 1 of this invention, which incorporates the standard cups 2 and 3, in addition to the shoulder straps 4 and 5, and a strap that locates around the back, as at 6, and having its usual fastener 7 located therein. Structured within this bra, as will be subsequently described, and preferably within an openable pouch, as noted at 8 and 9, are the individual suction cups 10, which may locate within the pouches 8 or 9 during usages. There may be 2 such suction cups 10 employed. See FIG. 2. FIG. 3 discloses a milk reservoir 11, which also fits within one of the pouches 8 or 9, and for use for the collection of milk from the mother, when time requires as such. It can be seen that the reservoir has a connector 12 to which the various tubing operatively associated with this invention may connect, and then likewise, such tubing may connect with the integrated connector 13, operatively associated with each suction cup 10.

FIG. 2 discloses the micropump 14 of this invention. Such a micropump may be obtained from KNF Global Company, located at 2 Black Forest Road, Trenton, N.J. 08691, under Model No. WMS020, identified as the High-Efficiency Micropump. Such a micropump incorporates a small pump, preferably may be battery operated, and such a battery may be rechargeable, to provide for the operation of the micropump, when turned on. The pump may incorporate an off/on switch, and a shut off valve as at 15, and it may have a tube connector 16 that provides for the interconnection of tubing, for pumping purposes, with the connector 17 functionally provided upon the suction cup 10, as can be noted. Hence, when the pump is operative, it provides for the expression of milk from the mothers breast, an it's passing through tubing from the outlet 13, to the inlet 12, of the milk reservoir 11, for collection purposes. As noted, the reservoir may be designed for removable insertion within the pouches 8 or 9, when used, or the reservoir may be located externally, and connected by tubing, for operative purposes, for the collection of the expressed milk.

Obviously, any battery provided within the micropump 14 will desirably be rechargeable. FIG. 2 discloses the bra 1 of this invention, and shows the location of the micropump 14 intermediate the bra cups 2 and 3, with the individual suction cups 10, one operatively associated with each of the bra cups, and which connect by tubing 18 between the pump and the suction cups, and through further tubing 19 to the shown reservoirs 11.

FIG. 3 shows how the suction cups 10 are of thin line construction may be made of a latex or polymer, or easily cleansable, and which have the various tubing ports 18 and 19 integrated therewith. The cup has an arcuate shape to provide for its conformity when locating upon the breast of the mother.

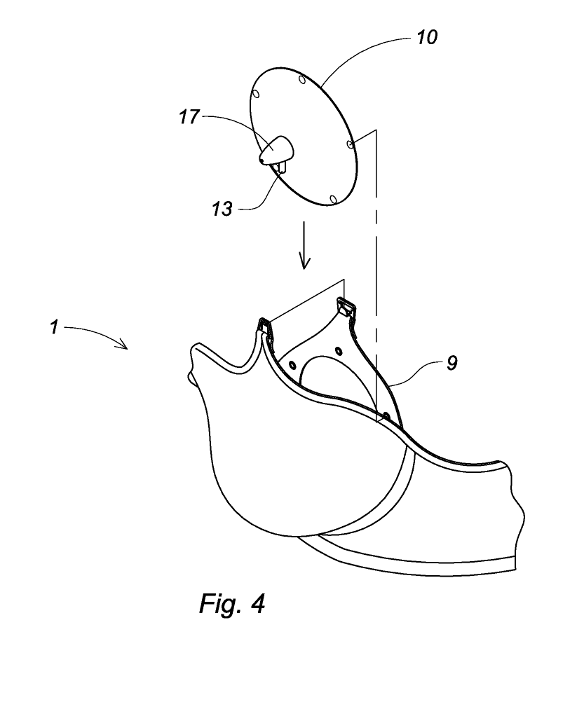

FIG. 4 shows the provision of the pouches 8 or 9 that are formed within the bra, and which may accommodate the locating of one of the suction cups 10 therein, and which discloses the tubing connector, as at 20 for connecting with tubing that leads to the pump, and in addition, shows the connector 21 for connecting with the tubing 19 for securement with one of the reservoirs 11 for fluid connecting purposes.

FIG. 2 shows the approximate length and size of the type of tubing 18 and 19 that may be used for interconnecting between the micropump, and the suction cup, when the structured bra is assembled for usage.

FIG. 3 shows the configuration of a reservoir 11, with its connector 12, and as can be seen, the reservoir has an arcuate shape, so that it can be easily accommodated within a pouch 8 formed within the bra, when it is desired to function as a reservoir for collection of any expressed milk. Or, as stated, the reservoir may be located externally, for the convenience of the mother, depending upon the circumstances under which mother's milk is being collected for further usage.

FIG. 2 discloses the micropump 14 of this invention. It can be obtained as previously described from its supplier. It has the identified off/on switch 15 all embodied upon the encasement 21, it may have a D/C or A/C chargeable connecting port 22, and has the various tubing connectors 16 provided thereon. The micropump is of miniature size, so that it conveniently can fit either within the bra, or externally thereof, between the shown cups 2 and 3, during its application and usage. See also FIG. 1.

What is important is that the concept of this invention integrates directly within a brassiere, one that can function almost under normal condition, and look no different than the standard bra worn by any women. See FIG. 8. But, all the integrated components, as previously described, can be embodied therein, within

I claim:

1. A brassiere that functions as a breast pump for a women, where breast milk may need to be pumped at select times of the day regardless of the surrounding circumstances, the brassiere incorporates various cups, and straps, that provide for adherence of the brassiere to the mother, a pump integrated within the structure of the brassiere, preferably intermediate the pair of cups, at least one suction cup associated with at least one of the bra cups, and at least one reservoir associated with one of the cups, tubing interconnecting the micro pump to the suction cups, further tubing connecting the suction cup to said reservoir, so that when the pump is actuated, milk may be expressed by the mother through the suction cup to the reservoir for collection.

2. The breast pump brassiere of claim 1 wherein the pump is a micro pump, and the micro pump, suction cup, and reservoir, are operatively structured to each breast cup, to function for collection of expressed milk and its storage within an associated milk reservoir.

3. The breast pump brassiere of claim 2 wherein each cup of the brassiere has a pouch for locating of at least a suction cup therein, during usage and operation of the brassiere.

4. The breast pump brassiere of claim 3 wherein the reservoir may locate within the pouch, or externally thereof, and connect by tubing to the suction cup, during usage.

5. The breast pump brassiere of claim 4 and including further tubing interconnected between the micro pump, and each one of the suction cups, to provide for partial vacuuming for drawing any mothers' milk for collection within its associated reservoir.

6. The breast pump brassiere of claim 5, wherein the pouches are open externally of each cup to facilitate the introduction and removal of any suction cup and reservoir.
